(19) United States
(12) Patent Application Publication
(10) Pub. No.: US 2025/0382276 A1
(43) Pub. Date: Dec. 18, 2025

(54) COMPOUND, METHOD OF PREPARATION, COMPOSITION, AND USES THEREOF

(71) Applicant: AMRITA SCHOOL OF PHARMACY, AMRITA VISHWA VIDYAPEETHAM, Kochi (IN)

(72) Inventors: Bijo Mathew, Kochi (IN); Sunil Kumar, Kochi (IN)

(73) Assignee: AMRITA SCHOOL OF PHARMACY, AMRITA VISHWA VIDYAPEETHAM, Kochi (IN)

(21) Appl. No.: 19/241,703

(22) Filed: Jun. 18, 2025

(30) Foreign Application Priority Data
Jun. 18, 2024 (IN) 202441046704

(51) Int. Cl. C07D 317/54; A61K 31/343; A61K 31/36; A61K 31/381; A61K 31/404; C07D 209/12; C07D 307/80; C07D 333/22; C07D 333/28

(52) U.S. Cl. CPC C07D 317/54; A61K 31/343; A61K 31/36; A61K 31/381; A61K 31/404; C07D 209/12; C07D 307/80; C07D 333/22; C07D 333/28

(57) ABSTRACT

Compounds that are monoamine oxidase-B inhibitors, a process for preparing the compounds and a composition comprising the compounds. The compounds are useful for the treatment of neurological disorders.

### CROSS-REFERENCED APPLICATION

This application claim priority to Indian Patent Application number 202441046704, filed Jun. 18, 2024, which is incorporated herein in its entirety by reference thereto.

### FIELD OF THE DISCLOSURE

The present disclosure relates to compounds that are monoamine oxidase-B inhibitors. The present disclosure also provides a process for preparing the compounds and a composition comprising the compounds. The compounds of the present disclosure are useful for the treatment of neurological disorders.

### BACKGROUND OF DISCLOSURE

A neurological or neurogenerative disorder is any condition affecting the nervous system. This disorder is caused by the progressive loss of structure or function of neurons. These structural, biochemical, or electrical abnormalities in the brain, spinal cord, or other nerves can cause various symptoms, including paralysis, muscle weakness, poor coordination, loss of sensation, seizures, confusion, pain, tauopathies, and altered consciousness. There are many recognized neurological disorders, ranging from common to rare. Among these disorders, Parkinson's disease (PD) stands as one of the most prevalent neurodegenerative disorders globally, characterized by progressive motor dysfunction, including tremors, bradykinesia, rigidity, and postural instability. Current PD treatments aim to alleviate motor impairments and restore dopamine function. Strategies include using medications like L-DOPA (L-dihydroxyphenylalanine), also known as levodopa, to replace dopamine, blocking dopamine reuptake, and targeting post-synaptic dopamine receptors with direct agonists.

PD is linked to unbalanced levels of Monoamine oxidase (MAOs) in the brain. Monoamine oxidases (MAOs) are enzymes that regulate neurotransmitter levels. These enzymes are primarily located on the outer membrane of mitochondria in various cells throughout the body and have an important role in the metabolism of neuroactive monoamines, such as dopamine (DA), norepinephrine, serotonin, and melatonin, in the brain. There are two distinct types of MAO enzymes: MAO-A and MAO-B.

MAO-B is an enzyme which is encoded by the MAOB gene in humans. MAO-B's normal activity generates reactive oxygen species, damaging cells directly. With age, MAO-B levels rise, possibly contributing to age-related cognitive decline and increased susceptibility to neurological diseases later in life. More active MAO-B gene variations are associated with negative emotions and implicated in depression. Increased MAO-B levels in the brain are connected to amyloid B-peptide (AB) accumulation, contributing to plaque formation. This elevated level of MAO-B enzyme implicated in neurological disorders like Parkinson's and Alzheimer's disease, emphasizes the importance of MAO-B inhibitors in managing these conditions effectively. These MAO-B inhibitors increase dopamine availability in the brain, thereby enhancing motor symptoms and potentially slowing disease progression. These inhibitors are particularly important in the management of PD, either alone or in conjunction with other medicines, due to their impact on dopaminergic activity.

While irreversible MAO-B inhibitors have been used therapeutically, they possess drawbacks such as potential interference with other targets and immunogenicity. the potential to interfere with other targets, the immunogenicity of the MAO-B-inhibitor complex, a longer half-life, a decreased sensitivity to ADME (absorption, distribution, metabolism, and excretion) measurements, and the potential for the brain to produce new MAO enzymes in response to inhibition.

The Food and Drug Administration (FDA) and the European Union (EU) recently approved safinamide, a reversible MAO-B inhibitor, for mid- to late-stage PD treatment.

Although the Parkinson's disease (PD) medications now on the market significantly reduce symptoms, they have a variety of negative side effects. Treating non-motor symptoms, regulating uneven pharmaceutical efficaciousness, and managing long-term side effects are some of these concerns. The adverse effects of current Parkinson's disease treatments frequently include dry mouth, lightheadedness, nausea, constipation, diarrhea, drowsiness, and/or dizziness.

In view of above challenges, there is a continuing need for improved compounds, compositions and methods that can provide continuous and consistent dopamine levels in the brain to effectively treat movement disorders such as Parkinson's disease.

In this way, the inventors of the present disclosure found that cinnamyl moiety in one scaffold can potentially enhance the MAO-B binding affinity via the formation of ionic interactions, hydrogen bonds and n-interactions with amino acid residues in the active site of the MAO-B. Thus, the inventors have made a surprising finding by providing heterocyclic-derived conjugated dienones compounds as selective MAO-B inhibitors.

### Objective of the Disclosure

The primary objective of the present disclosure is to provide compounds that are monoamine oxidase-B inhibitors.

Another objective of the present disclosure is to provide a pharmaceutical composition comprising the compounds that are monoamine oxidase-B inhibitors.

Another objective of the present disclosure is to provide a process for preparing a compound of the disclosure.

Another objective of the present disclosure is to provide the compounds for use in the treatment of neurological or neurodegenerative disorders.

Another objective of the present disclosure is to provide the pharmaceutical compositions for use in the treatment of neurological or neurodegenerative disorders.

Another objective of the present disclosure is to provide the compounds or the pharmaceutical compositions for use in the treatment of Parkinson disease.

### SUMMARY

The present disclosure provides a compound of Formula (I):

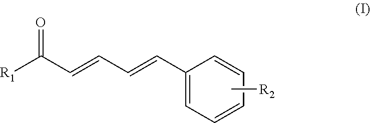

or a pharmaceutically acceptable salt thereof;
wherein R₁ is selected from the group comprising C₆-C₁₄ aryl, 5-14 membered heterocyclyl, and 5-14 membered heteroaryl, wherein aryl, heterocyclyl, heteroaryl, are optionally substituted with groups selected from hydrogen, halogen, C₁-C₆ alkyl, alkoxy, and —NO₂; and
R₂ is selected from hydrogen, halogen, C₁-C₆ alkyl, alkoxy, and —NO₂.

In another aspect, the present disclosure provides the compounds of formula (I) that are monoamine oxidase-B inhibitors.

The present disclosure provides a pharmaceutical composition comprising the compounds of formula (I).

The present disclosure provides a process for preparing the compounds of formula (I).

The present disclosure provides the compounds of formula (I) for use in the treatment of neurological or neurodegenerative disorder in a subject.

The present disclosure provides the pharmaceutical composition for use in the treatment of neurological or neurodegenerative disorder in a subject.

The present disclosure provides the compounds of formula (I) or the pharmaceutical compositions for use in the treatment of Parkinson's disease (PD).

The present disclosure provides a method of treating or preventing a disease, disorder or condition in a subject comprising administering to the subject a therapeutically effective amount of a compound of formula (I) described herein.

### BRIEF DESCRIPTION OF DRAWINGS

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure where in:

FIG. 1 depicts the concentration dependent inhibition of MAO-B by Compounds 11 (A) and 12 (B). The results were expressed as mean±SD of triplicate experiments.

FIG. 2 depicts Lineweaver-Burk (LB) plots for MAO-B inhibition of Compound 14 (FIG. 2A) and Compound 11 (FIG. 2C), and respective secondary plots (i.e., slopes vs inhibitors concentration) of Compound 14 (FIG. 2B) and Compound 11 (FIG. 2D). The experiments were carried out at five different concentrations of a substrate benzylamine and three different concentrations of Compound 11 or Compound 14. These results were expressed as mean±SD of triplicate experiments.

FIG. 3 depicts the recovery of MAO-B inhibition by Compound 11 and Compound 14 using dialysis experiments. The concentrations of Compound 11 and Compound 14 were approximately double their IC₅₀ values. After 30 minutes of preincubation, the mixtures were dialyzed for 6 hours with buffer change at 3-hours interval. The results were expressed as mean±SD of triplicate experiments.

### DETAILED DESCRIPTION OF THE PREFERRED EMBODIMENT

At the very outset of the detailed description, it may be understood that the ensuing description only illustrates a particular form of this disclosure. However, such a particular form is only an exemplary embodiment, and without intending to imply any limitation on the scope of this disclosure. Accordingly, the description is to be understood as an exemplary embodiment and teaching of disclosure and not intended to be taken restrictively.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

The term “alkyl” refers to a straight or branched chain saturated aliphatic hydrocarbon that may be substituted or unsubstituted. In certain embodiments, the alkyl is C₁-C₆ alkyl. Examples of “alkyl” include but are not limited to methyl, ethyl, n-propyl, isopropyl, n-butyl, t-butyl, n-pentyl, isobutyl and the likes thereof.

The term “alkoxy” refers to a group-O-alkyl, wherein alkyl is as defined above. Representative examples include, but are not limited to, methoxy, ethoxy, propoxy, t-butoxy and the likes thereof.

The term “aryl” refers to (i) optionally substituted phenyl, (ii) optionally substituted 9- or 10-membered bicyclic, fused carbocyclic ring systems in which at least one ring is aromatic, and (iii) optionally substituted 11- to 14-membered tricyclic, fused carbocyclic ring systems in which at least one ring is aromatic. Suitable aryls include, for example, phenyl, biphenyl, naphthyl, tetrahydronaphthyl (tetralinyl), indenyl, anthracenyl, and fluorenyl.

The term “phenyl” as used herein is meant to indicate that optionally substituted or non-substituted phenyl group.

As used herein, the term ‘compound(s)’ comprises the compounds disclosed in the present disclosure.

As used herein, the term “comprises” or “comprising” is generally used in the sense of include, that is to say permitting the presence of one or more features or components.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Compounds of the disclosure” or “present disclosure” refers to the compounds of (I), as herein defined, or a pharmaceutical salt thereof, a composition containing them. The compounds of the present disclosure may be useful as monoamine oxidase-B inhibitors particularly for treating Parkinson's disease.

The term “halogen” refers to fluorine, chlorine, bromine, or iodine.

As used herein, the term “heteroaryl” refers to monocyclic aromatic ring systems or fused bicyclic aromatic ring systems comprising two or more aromatic rings, preferably two to three ring systems. These heteroaryl rings contain one or more nitrogen, sulfur and/or oxygen atoms where N-oxides sulfur oxides and dioxides are permissible heteroatom substitutions. The term includes ring(s) optionally substituted with halogens, nitro, amino, alkoxy, alkyl sulfonyl amino, alkylcarbonylamino, carboxy, alkyl carbonoyl, hydroxy, and alkyl. Examples of heteroaryl groups include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, chromanyl, isochromanyl and the likes thereof.

The term “heterocyclyl” refers to a stable 3 to 15 membered ring that is either saturated or has one or more degrees of unsaturation or unsaturated. These heterocyclic rings contain one or more heteroatoms selected from the group consisting of nitrogen, sulfur, and oxygen where N-oxides, sulfur oxides and dioxides are permissible heteroatom substitutions. Such a ring is optionally fused to one or more of another heterocyclic ring(s), aryl ring(s) or cycloalkyl ring(s). Examples of such groups are selected from the group consisting of azetidinyl, acridinyl, pyrazolyl, imidazolyl, triazolyl, pyrrolyl, thiophenyl, thiazolyl, oxazolyl, isoxazolyl, furanyl, pyrazinyl, tetrahydroisoquinolinyl, piperidinyl, piperazinyl, morpholinyl, thiomorphonilyl, pyridazinyl, indolyl, isoindolyl, quinolinyl, chromanyl and the likes thereof. “Heterocyclylalkyl” refers to a heterocyclic ring radical defined above, directly bonded to an alkyl group. The heterocyclylalkyl radical is attached to the main structure at carbon atom in the alkyl group that results in the creation of a stable structure.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

A “pharmaceutically acceptable salt” refers to the salts of the compounds, that is pharmaceutically acceptable and that possesses the desired pharmacological activity of the parent compound. Pharmaceutically acceptable salts of the compounds of this disclosure include those derived from suitable inorganic and organic acids and bases. Such salts include: salts derived from inorganic bases such as Li, Na, K, Ca, Mg, Fe, Cu, Zn, Al, Mn; salts of organic bases such as N,N′-diacetylethylenediamine, 2-dimethylaminoethanol, isopropylamine, morpholine, piperazine, piperidine, procaine, diethylamine, triethylamine, trimethylamine, tripropylamine, tromethamine, choline hydroxide, dicyclohexylamine, metformin, benzylamine, phenylethylamine, dialkylamine, trialkylamine, thiamine, aminopyrimidine, aminopyridine, purine, pyrimidine, spermidine, and the like; chiral bases like alkylphenylamine, glycinol, phenyl glycinol and the like, salts of natural amino acids such as glycine, alanine, valine, leucine, isoleucine, lysine, arginine, serine, threonine, phenylalanine; unnatural amino acids such as D-isomers or substituted amino acids; salts of acidic amino acids such as aspartic acid, glutamic acid; guanidine, substituted guanidine wherein the substituents are selected from nitro, amino, alkyl, alkenyl, alkynyl, ammonium or substituted ammonium salts. Salts may include acid addition salts where appropriate which are sulphates, nitrates, phosphates, perchlorates, borates, hydrohalides, acetates, tartrates, maleates, citrates, succinates, methanesulfonates, benzoates, salicylates, hydroxynaphthoates, benzenesulfonates, ascorbates, and the likes thereof.

For therapeutic use, salts of active ingredients of the compounds of the disclosure will typically be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived from a physiologically acceptable acid or base, are within the scope of the present disclosure.

The term “pharmaceutically acceptable carrier” denotes any inactive substance or blend of substances employed in making a pharmaceutical formulation. Its purpose is to help in the delivery, absorption, stability, or administration of an active pharmaceutical ingredient (API) to a patient. Carriers can be in the form of solids, liquids, or gases and are generally pharmacologically inert, ensuring they do not disrupt the therapeutic actions of the API. Various examples of pharmaceutical carriers comprise diluents, binders, disintegrants, fillers, solvents, preservatives, emulsifiers, and excipients.

Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The pharmaceutical composition of the present disclosure can be administered to a subject via various routes, including orally (e.g., drenches in aqueous or non-aqueous solutions or suspensions, tablets, capsules including sprinkle and gelatin capsules, boluses, powders, granules, and pastes for tongue application); through oral mucosa absorption (e.g., sublingually); anally, rectally, or vaginally (e.g., as a pessary, cream, or foam); parenterally (e.g., intramuscularly, intravenously, subcutaneously, or intrathecally as a sterile solution or suspension); nasally; intraperitoneally; transdermally (e.g., as a patch applied to the skin); and topically (e.g., as a cream, ointment, or spray applied to the skin, or as eye drops).

The compositions can be conveniently presented in unit dosage form and may be prepared by methods well-known in pharmacy. The dosage form can be presented in solid form or liquid form. The amount of active ingredient combined with a carrier material to produce a single dosage form will vary depending on the host being treated and the mode of administration. Compositions suitable for oral administration may come in the form of capsules (including sprinkle and gelatin capsules), cachets, pills, tablets, lozenges (with a flavored base, usually sucrose and acacia or tragacanth), lyophiles, powders, granules, or as a solution or suspension in an aqueous or non-aqueous liquid, an oil-in-water or water-in-oil emulsion, an elixir or syrup, pastilles (using an inert base such as gelatin and glycerin or sucrose and acacia), and/or mouthwashes.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate and/or any of the following: (1) fillers or extenders, such as starches, lactose, sucrose, glucose, mannitol and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate and mixtures thereof; (10) complexing agents, such as, modified and unmodified cyclodextrins; and (11) coloring agents.

Liquid dosage forms useful for oral administration include pharmaceutically acceptable emulsions, lyophiles for reconstitution, microemulsions, suspensions, solutions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

The pharmaceutical composition of the present disclosure may be used alone (monotherapy) or conjointly with one or more other methods/compositions (combined therapy).

Unless otherwise specified, the term “substituted” as used herein refers to mono, bi, tri or tetra substitution with any one or combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR₂, —C(O)R₂, —C(S)R₂, —C(O)NR₂R₃, —C(O)ONR₃R₄, —NR₂CONR₃R₄, —N(R₂)SOR₃, —N(R₂)SO₂R₃, —(═N—N(R₂)R₃), —NR₂C(O)OR₃, —NR₂R₃, —NR₂C(O)R₃, —NR₂C(S)R₃, —NR₂C(S)NR₃R₄, —SONR₂R₃, —SO₂NR₂R₃, —OR₂, —OR₂C(O)NR₃R₄, —OR₂C(O) OR₃, —OC(O)R₂, —OC(O)NR₂R₃, —R₂NR₃C(O)R₄, —R₂OR₃, —R₂C(O) OR₃, —R₂C(O)NR₃R₄, —R₂C(O)R₃, —R₂OC(O)R₂, —SR₂, —SOR₂, —SO₂R₂, and —ONO₂, wherein R₂, R₃ and R₄ are independently selected from hydrogen, substituted or unsubstituted alkyl, substituted or unsubstituted alkoxy, substituted or unsubstituted alkenyl, substituted or unsubstituted alkynyl, substituted or unsubstituted alkylene, substituted or unsubstituted alkenylene, substituted or unsubstituted alkynylene, substituted or unsubstituted aryl, substituted or unsubstituted arylene, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted or unsubstituted heteroarylene, substituted or unsubstituted heterocyclylene, substituted heterocyclylalkyl ring, substituted or unsubstituted heteroarylalkyl, or substituted or unsubstituted heterocyclic ring. Alternately R₃ and R₄ together along with the nitrogen they are attached to, form a 4 to 8 membered ring which can be substituted or unsubstituted.

As used herein, the term “therapeutically effective amount” refers to that amount of the compound being administered sufficient to prevent development of or alleviate to some extent one or more of the symptoms of the condition or disorder being treated.

As used herein, the term “treat”, “treating” and “treatment” refer to a method of alleviating or abrogating a disease and/or its attendant symptoms.

Each embodiment is provided by way of explanation of the disclosure and not by way of limitation of the disclosure. In fact, it will be apparent to those skilled in the art that various modifications and variations can be made to the compounds, compositions and methods described herein without departing from the scope or spirit of the disclosure. For instance, features illustrated or described as part of one embodiment can be applied to another embodiment to yield a still further embodiment. Thus, it is intended that the present disclosure includes such modifications and variations and their equivalents. Other objects, features and aspects of the present disclosure are disclosed in or are obvious from, the following detailed description. It is to be understood by one of ordinary skill in the art that the present discussion is a description of exemplary embodiments only and is not to be construed as limiting the broader aspects of the present disclosure.

In an embodiment, the present disclosure provides a compound of Formula (I):

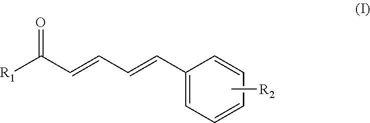

or a pharmaceutically acceptable salt thereof;
R₁ is selected from a group comprising C₆-C₁₄ aryl, 5-14 membered heterocyclyl, 5-14 membered heteroaryl, wherein aryl, heterocyclyl, and heteroaryl, are optionally substituted with a group selected from hydrogen, halogen, C₁-C₆ alkyl, C₁-C₆ alkoxy, and —NO₂; and
R₂ is selected from a group comprising hydrogen, halogen, C₁-C₆ alkyl, C₁-C₆ alkoxy, and —NO₂.

In certain embodiments, R₁ is C₆-C₁₄ aryl.

In certain embodiments, R₁ is 5-14 membered heterocyclyl.

In some embodiments, R₁ is 5 membered heterocyclyl, 6 membered heterocyclyl, 7 membered heterocyclyl, 8 membered heterocyclyl, 9 membered heterocyclyl, 10 membered heterocyclyl, 11 membered heterocyclyl, 12 membered heterocyclyl, 13 membered heterocyclyl, or 14 membered heterocyclyl.

In certain embodiments, R₁ is 5-14 membered heteroaryl.

In some embodiments, R₁ is selected from 5 membered heteroaryl, 6 membered heteroaryl, 7 membered heteroaryl, 8 membered heteroaryl, 9 membered heteroaryl, 10 membered heteroaryl, 11 membered heteroaryl, 12 membered heteroaryl, 13 membered heteroaryl, and 14 membered heteroaryl.

In a specific embodiment, R₁ is thiophene.

In a specific embodiment, R₁ is indole.

In a specific embodiment, R₁ is benzofuran.

In a specific embodiment, R₁ is benzodioxol.

In certain embodiments, R₁ is substituted with halogen.

In some embodiments, R₁ is substituted with a group selected from fluorine, chlorine, bromine, and iodine.

In some embodiments, R₁ is substituted with fluorine.

In some embodiments, R₁ is substituted with chlorine.

In some embodiments, R₁ is substituted with bromine.

In some embodiments, R₁ is substituted with iodine.

In certain embodiments, R₁ is substituted with C₁-C₆ alkyl.

In some embodiments, R₁ is substituted with a group selected from methyl, ethyl, propyl, butyl, pentyl, and hexyl.

In certain embodiments, R₁ is substituted with alkoxy.

In some embodiments, R₁ is substituted with a group selected from methoxy, ethoxy, propoxy, or butoxy.

In certain embodiments, R₂ is hydrogen.

In certain embodiments, R₂ is halogen.

In some embodiments, R₂ is selected from a group comprising fluorine, chlorine, bromine, and iodine.

In some embodiments, R₂ is fluorine.

In some embodiments, R₂ is chlorine.

In some embodiments, R₂ is bromine.

In some embodiments, R₂ is iodine.

In certain embodiments, R₂ is C₁-C₆ alkyl.

In some embodiments, R₂ is selected from methyl, ethyl, propyl, butyl, pentyl, and hexyl.

In certain embodiments, R₂ is alkoxy.

In some embodiments, R₂ is selected from methoxy, ethoxy, propoxy, and butoxy.

In a specific embodiment, R₂ is methoxy.

In some embodiments, R₂ is ethoxy.

In some embodiments, R₂ is propoxy.

In some embodiments, R₂ is butoxy.

In a specific embodiment, R₂ is —NO₂.

In an embodiment, the present disclosure provides a compound of Formula (Ia):

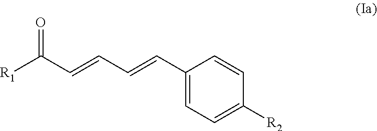

or a pharmaceutically acceptable salt thereof;
wherein R₁ and R₂ have the same meanings as defined for general formula (I) above.

In certain embodiments, the present disclosure provides a compound selected from the following list provided in Table 1.

TABLE 1
A list of compounds and their molecular weight:

| Compounds | | |
|---|---|---|
| No. (CD) | Structure | Molecular Weight |
| 1. | 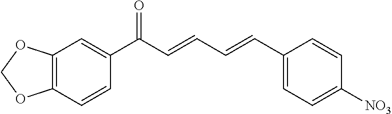 | 323.30 |
| 2. | 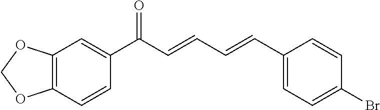 [SMILES: O=C(/C=C/C=C/c1ccc(Br)cc1)c1ccc2c(c1)OCO2] | 357.20 |
| 3. | 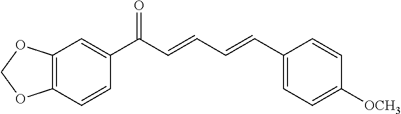 [SMILES: COc1ccc(/C=C/C=C/C(=O)c2ccc3c(c2)OCO3)cc1] | 308.33 |
| 4. | 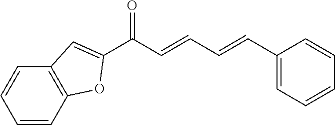 [SMILES: O=C(/C=C/C=C/c1ccccc1)c1cc2ccccc2o1] | 274.32 |
| 5. | 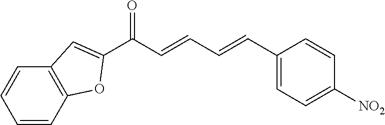 [SMILES: O=C(/C=C/C=C/c1ccc([N+](=O)[O-])cc1)c1cc2ccccc2o1] | 319.32 |
| 6. | 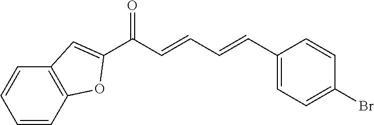 [SMILES: O=C(/C=C/C=C/c1ccc(Br)cc1)c1cc2ccccc2o1] | 353.22 |
| 7. | 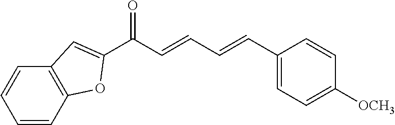 [SMILES: COc1ccc(/C=C/C=C/C(=O)c2cc3ccccc3o2)cc1] | 304.35 |
| 8. | 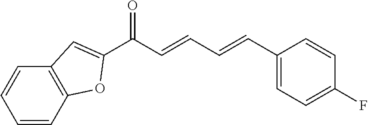 [SMILES: O=C(/C=C/C=C/c1ccc(F)cc1)c1cc2ccccc2o1] | 292.31 |
| 9. | 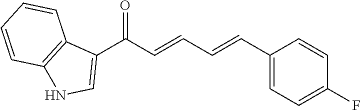 [SMILES: O=C(/C=C/C=C/c1ccc(F)cc1)c1c[nH]c2ccccc12] | 291.33 |
| 10. | 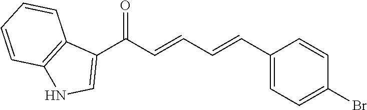 [SMILES: O=C(/C=C/C=C/c1ccc(Br)cc1)c1c[nH]c2ccccc12] | 352.23 |
| 11. | 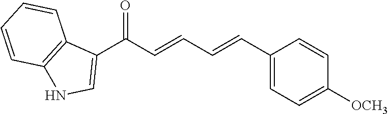 [SMILES: COc1ccc(/C=C/C=C/C(=O)c2c[nH]c3ccccc23)cc1] | 303.36 |
| 12. | 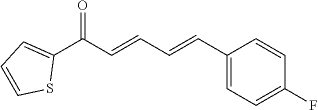 [SMILES: O=C(/C=C/C=C/c1ccc(F)cc1)c1cccs1] | 258.31 |
| 13. | 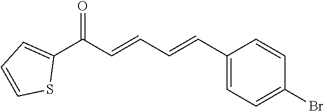 [SMILES: O=C(/C=C/C=C/c1ccc(Br)cc1)c1cccs1] | 319.22 |
| 14. | 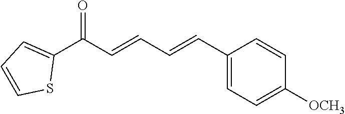 [SMILES: COc1ccc(/C=C/C=C/C(=O)c2cccs2)cc1] | 270.35 |
| 15. | 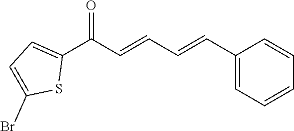 [SMILES: O=C(/C=C/C=C/c1ccccc1)c1ccc(Br)s1] | 319.22 |
| 16. | 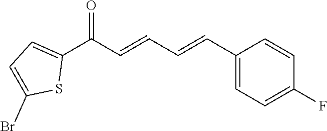 [SMILES: O=C(/C=C/C=C/c1ccc(F)cc1)c1ccc(Br)s1] | 337.21 |
| 17. | 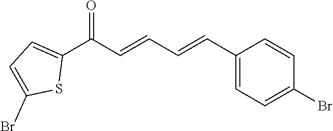 [SMILES: O=C(/C=C/C=C/c1ccc(Br)cc1)c1ccc(Br)s1] | 398.11 |
| 18. | 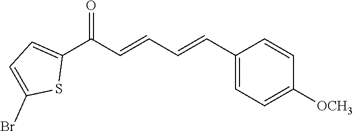 [SMILES: COc1ccc(/C=C/C=C/C(=O)c2ccc(Br)s2)cc1] | 349.24 |

In certain embodiments, the present disclosure provides a compound selected from the list as described above or a pharmaceutically acceptable salt thereof.

In certain embodiments, the present disclosure provides a pharmaceutical composition comprising compounds of formula (I) or formula (Ia).

In certain embodiments, pharmaceutical composition comprising a compound of formula (I) or formula (Ia) and a pharmaceutically acceptable carrier.

In an embodiment, the present disclosure provides a process for preparing compounds of formula (I) or formula (Ia).

The process comprises a step of preparing a solution of the appropriate cinnamaldehyde derivatives dissolved in alcohol, adding this solution to a heterocyclic acetophenones to form a mixture, incorporating catalyst into the mixture, adding ice cubes to the solution, and stirring it overnight to produce multi-conjugated ketones. The final product is then cleaned with water, filtered, and dried overnight.

In some embodiments, the present disclosure provides a process of preparing compounds of formula (I), wherein the process comprises a step of reacting a substituted cinnamaldehyde of formula (A) with a heterocyclic acetophenone of formula B in a solvent, in the presence of a catalyst, at a room temperature for a period ranging from 6 hours to 24 hours:

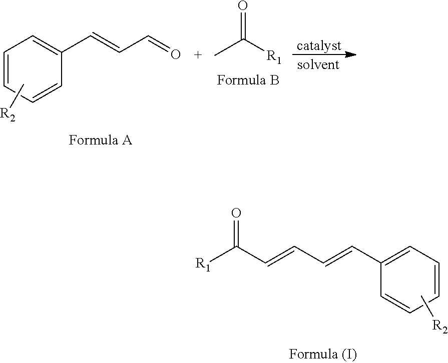

In some embodiments, the present disclosure provides a process of preparing compounds of formula (Ia), wherein the process comprises a step of reacting a substituted cinnamaldehyde of formula A₁ with a heterocyclic acetophenone of formula B₁ in a solvent, in the presence of a catalyst, at a room temperature for a period ranging from 6 hours to 24 hours:

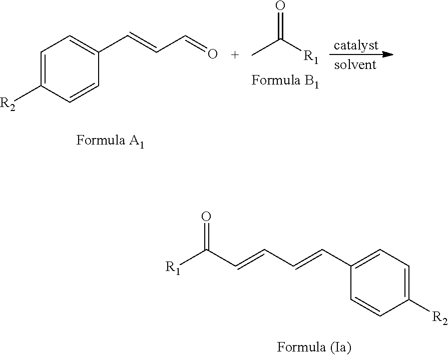

In some embodiments, the present disclosure provides a process of preparing compounds of formula (Ia), wherein the catalyst is pyrrolidine, and the solvent is selected from ethanol and methanol.

In certain embodiments, the present disclosure provides compounds of Formula (I) or (Ia) that are useful as monoamine oxidase-B inhibitors.

In certain embodiments, the present disclosure provides compounds of formula (I) or formula (Ia) that are useful for the treatment of neurological or neurodegenerative diseases, specifically Parkinson disease in a subject.

In certain embodiments, the subject is a mammal such as a human or a non-human mammal. In some embodiments, the subject is a human.

In certain embodiments, the present disclosure provides compounds of formula (I) or formula (Ia) for use in a medicament.

In certain embodiments, the present disclosure provides compounds of formula (I) or formula (Ia) for use in treating neurological disorders or neurodegenerative disorders that are selected from amyotrophic lateral sclerosis, multiple sclerosis, Parkinson's disease, Alzheimer's disease, Huntington's disease, multiple system atrophy, tauopathies, and prion diseases in a subject.

In some embodiments, the present disclosure provides compounds of formula (I) or formula (Ia) for use in treating Parkinson disease.

In certain embodiments, the present disclosure provides a method of treating a neurological or neurodegenerative disorder in a subject.

The method comprises administering to the subject a therapeutically effective amount of the compounds of formula (I) or formula (Ia) described herein.

In certain embodiments, the present disclosure provides a pharmaceutical composition comprising the compounds of formula (I) or formula (Ia) for use for use in treating neurological disorders or neurodegenerative disorders that are selected from amyotrophic lateral sclerosis, multiple sclerosis, Parkinson's disease, Alzheimer's disease, Huntington's disease, multiple system atrophy, tauopathies, and prion diseases in a subject.

In some embodiments, the present disclosure provides a pharmaceutical composition for use in treating Parkinson disease.

In some embodiments, the present disclosure provides a method of treating or preventing a disease, disorder or condition in a subject comprising administering to the subject a therapeutically effective amount of a compound of formula (I) described herein. The subject refers to an animal, particularly human.

In some embodiments, the present disclosure provides that the disease, disorder or condition is a neurological disorder or a neurodegenerative disorder.

In some embodiments, the present disclosure provides that the neurological disorder or the neurodegenerative disorder is selected from the group comprising amyotrophic lateral sclerosis, multiple sclerosis, Parkinson's disease, Alzheimer's disease, Huntington's disease, multiple system atrophy, tauopathies, and prion diseases.

In some embodiments, the present disclosure provides that the neurological disorder or the neurodegenerative disorder is Parkinson's disease (PD).

The present disclosure, now being generally described, will be more readily understood by reference to the following examples, which are included merely for purposes of illustration of certain aspects and embodiments of the present disclosure and are not intended to limit the present disclosure in any way.

### EXAMPLES

The following examples are given by way of illustration of the working of the disclosure in actual practice and therefore should not be construed to limit the scope of present disclosure.

The following chemicals and reagents were utilized in the experimental procedures described herein:
Recombinant MAO-A and MAO-B
Kynuramine
Benzylamine
Safinamide
Pargyline
Toloxatone
Clorgyline
   These compounds were procured from Sigma (St. Louis, MO, USA).
Anhydrous mono- and dibasic sodium phosphate were obtained from Daejung Chemicals and Metals (Siheung, South Korea).
A DiaEasy™ dialyzer (6-8 kDa) was sourced from BioVision (St. Grove MA, USA).

All other chemicals employed in this experiment were of analytical grade.

### General Scheme: Synthesis of Heterocyclic Conjugated Dienones:

The compounds were synthesized by pyrrolidine catalyzed reaction from substituted cinnamaldehyde as presented by following general scheme:

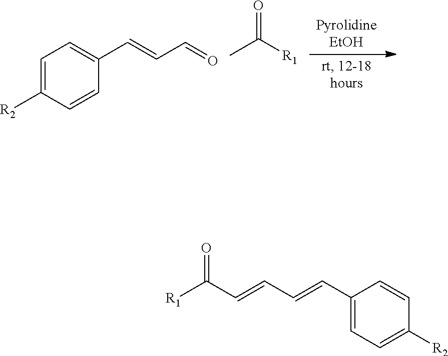

In this process, a stirred solution of the appropriate cinnamaldehyde derivatives (0.01 M) dissolved in 20 mL of ethanol was added to heterocyclic acetophenones (0.01 M). Without delay, pyrrolidine (0.01 M) was added to the mixture. When ice cubes were added to the solution, stirring it overnight produced multi-conjugated ketones. The final product was carefully cleaned with water, filtered using a suction cup, and then allowed to dry overnight in a desiccator.

Based on above scheme all the compounds of the present disclosure have been prepared, wherein R₁ and R₂ are specified as shown in below Table 2:

TABLE 2

| Compounds | | | | Characterization on data |
|---|---|---|---|---|
| No. | R₁ | R₂ | Structure & IUPAC name | (¹H NMR (500 MHz, CDCl₃) |
| 1 | benzodioxol | NO₂ | 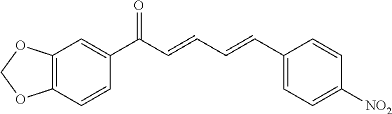 [SMILES: O=C(/C=C/C=C/c1ccc([N+](=O)[O-])cc1)c1ccc2c(c1)OCO2] | δ 7.59 (dd, J = 8.2, 1.7 Hz, 2H), 7.57-7.52 (m, 2H), 7.50 (d, J = 8.7 Hz, 5H), 7.36 (d, J = 8.5 Hz, 3H), 7.07 (d, J = 14.8 Hz, 3H), 7.00-6.93 (m, 2H), 6.89 (t, J = 8.9 Hz, 2H), 6.06 (s, 3H). |
| | | | (2E,4E)-1- | |
| | | | (benzo[d][1,3]dioxol-5- | |
| | | | yl)-5-(4- | |
| | | | nitrophenyl)penta-2,4- | |
| | | | dien-1-one | |
| 2 | benzodioxol | Br | 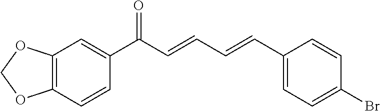 [SMILES: O=C(/C=C/C=C/c1ccc(Br)cc1)c1ccc2c(c1)OCO2] | δ 7.59 (dd, J = 8.2, 1.7 Hz, 2H), 7.57- 7.52 (m, 2H), 7.50 (d, J = 8.7 Hz, 5H), 7.36 (d, J = 8.5 Hz, 3H), 7.07 (d, J = 14.8 Hz, 3H), 7.00-6.93 (m, 2H), 6.89 (t, J = 8.9 Hz, 2H), 6.06 (s, 3H). |
| | | | (2E,4E)-1- | |
| | | | (benzo[d][1,3]dioxol-5- | |
| | | | yl)-5-(4- | |
| | | | bromophenyl)penta-2,4- | |
| | | | dien-1-one | |
| 3 | benzodioxol | OCH₃ | 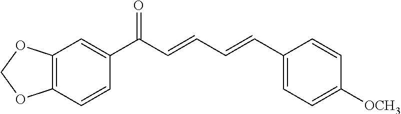 [SMILES: COc1ccc(/C=C/C=C/C(=O)c2ccc3c(c2)OCO3)cc1] | δ 7.58 (dd, J = 14.3, 11.2 Hz, 2H), 7.47 (dd, J = 23.7, 5.2 Hz, 3H), 6.98 (dd, J = 19.4, 15.2 Hz, 2H), 6.89 (dd, J = 12.7, 8.5 Hz, 4H), 6.05 (s, 2H), 3.84 (s, 3H). |
| | | | (2E,4E)-1- | |
| | | | (benzo[d][1,3]dioxol-5- | |
| | | | yl)-5-(4- | |
| | | | methoxyphenyl)penta- | |
| | | | 2,4-dien-1-one | |
| 4 | benzofuran | H | 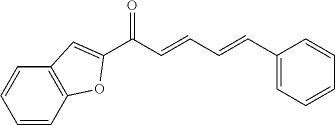 [SMILES: O=C(/C=C/C=C/c1ccccc1)c1cc2ccccc2o1] | δ 8.23 (d, J = 8.8 Hz, 2H), 7.73 (d, J = 7.9 Hz, 1H), 7.71-7.67 (m, 1H), 7.61 (t, J = 6.4 Hz, 3H), 7.55-7.48 (m, 2H), 7.33 (t, J = 7.6 Hz, 1H), 7.28-7.22 (m, 2H), 7.17 (dd, J = 15.6, 10.9 Hz, 1H), 7.07 (d, J = 15.6 Hz, 1H). |
| | | | (2E,4E)-1-(benzofuran-2- | |
| | | | yl)-5-phenylpenta-2,4- | |
| | | | dien-1-one | |
| 5 | benzofuran | NO₂ | 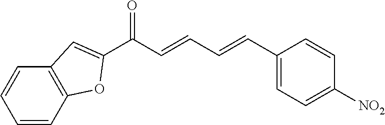 [SMILES: O=C(/C=C/C=C/c1ccc([N+](=O)[O-])cc1)c1cc2ccccc2o1] | δ 8.23 (d, J = 8.8 Hz, 2H), 7.73 (d, J = 7.9 Hz, 1H), 7.71-7.67 (m, 1H), 7.61 (t, J = 6.4 Hz, 3H), 7.55-7.48 (m, 2H), 7.33 (t, J = 7.6 Hz, 1H), 7.28-7.22 (m, 1H), 7.17 (dd, J = 15.6, 10.9 Hz, 1H), 7.07 (d, J = 15.6 Hz, 1H). |
| | | | (2E,4E)-1-(benzofuran-2- | |
| | | | yl)-5-(4- | |
| | | | nitrophenyl)penta-2,4- | |
| | | | dien-1-one | |
| 6 | benzofuran | Br | 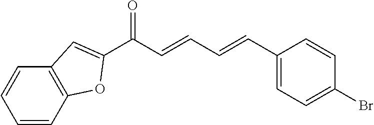 [SMILES: O=C(/C=C/C=C/c1ccc(Br)cc1)c1cc2ccccc2o1] | δ 7.76-7.66 (m, 2H), 7.64-7.57 (m, 2H), 7.54-7.45 (m, 3H), 7.37 (d, J = 8.5 Hz, 2H), 7.40-7.29 (m, 3H), 7.33 (dd, J = 11.5, 4.4 Hz, 1H), 7.16 (d, J = 15.0 Hz, 1H), 7.08-6.95 (m, 2H). |
| | | | (2E,4E)-1-(benzofuran-2- | |
| | | | yl)-5-(4- | |
| | | | bromophenyl)penta-2,4- | |
| | | | dien-1-one | |
| 7 | benzofuran | OCH₃ | 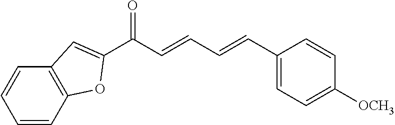 [SMILES: COc1ccc(/C=C/C=C/C(=O)c2cc3ccccc3o2)cc1] | δ 7.76-7.69 (m, 2H), 7.59 (dd, J = 18.3, 5.0 Hz, 2H), 7.47 (t, J = 8.3 Hz, 3H), 7.31 (t, J = 7.5 Hz, 1H), 7.10 (d, J = 14.9 Hz, 1H), 7.03 (d, J = 15.5 Hz, 1H), 6.93 (dd, J = 16.7, 10.0 Hz, 3H), 3.84 (s, 3H). |
| | | | (2E,4E)-1-(benzofuran-2- | |
| | | | yl)-5-(4- | |
| | | | methoxyphenyl)penta- | |
| | | | 2,4-dien-1-one | |
| 8 | benzofuran | F | 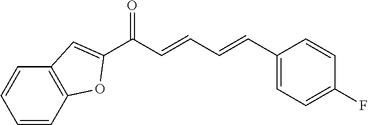 [SMILES: O=C(/C=C/C=C/c1ccc(F)cc1)c1cc2ccccc2o1] | δ 7.75-7.68 (m, 2H), 7.63-7.57 (m, 2H), 7.49 (dd, J = 7.9, 4.4 Hz, 3H), 7.32 (t, J = 7.5 Hz, 1H), 7.14 (d, J = 15.0 Hz, 1H), 7.07 (dd, J = 16.8, 8.2 Hz, 2H), 7.02-6.92 (m, 2H). |
| | | | (2E,4E)-1-(benzofuran-2- | |
| | | | yl)-5-(4- | |
| | | | fluorophenyl)penta-2,4- | |
| | | | dien-1-one | |
| 9 | indole | F | 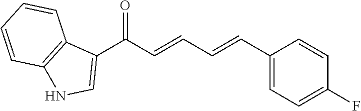 [SMILES: O=C(/C=C/C=C/c1ccc(F)cc1)c1c[nH]c2ccccc12] | δ 8.79 (s, 1H), 8.44-8.35 (m, 1H), 7.87 (d, J = 3.0 Hz, 2H), 7.49 (d, J = 8.5 Hz, 1H), 7.43-7.42 (m, 1H), 7.41 (dd, J = 3.2, 1.0 Hz, 1H), 7.34 (d, J = 8.5 Hz, 1H), 7.31- 7.28 (m, 3H), 6.92 (d, J = 8.5 Hz, 1H), 6.57 (d, J = 15.2 Hz, 1H). |
| | | | (2E,4E)-5-(4- | |
| | | | fluorophenyl)-1-(1H- | |
| | | | indol-3-yl)penta-2,4- | |
| | | | dien-1-one | |
| 10 | indole | Br | 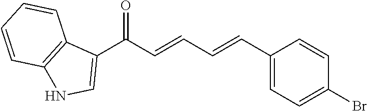 [SMILES: O=C(/C=C/C=C/c1ccc(Br)cc1)c1c[nH]c2ccccc12] | δ 8.79 (s, 1H), 8.51-8.28 (m, 1H), 7.87 (d, J = 3.0 Hz, 2H), 7.55-7.38 (m, 3H), 7.36- 7.28 (m, 4H), 6.92 (d, J = 8.5 Hz, 1H), 6.57 (d, J = 15.2 Hz, 1H). |
| | | | (2E,4E)-5-(4- | |
| | | | bromophenyl)-1-(1H- | |
| | | | indol-3-yl)penta-2,4- | |
| | | | dien-1-one | |
| 11 | indole | OCH₃ | 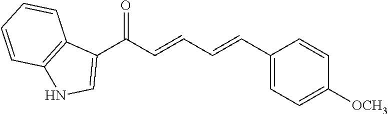 [SMILES: COc1ccc(/C=C/C=C/C(=O)c2c[nH]c3ccccc23)cc1] | δ 7.77-7.68 (m, 2H), 7.59 (dd, J = 18.3, 5.0 Hz, 2H), 7.47 (t, J = 8.3 Hz, 3H), 7.31 (t, J = 7.5 Hz, 1H), 7.10 (d, J = 14.9 Hz, 1H), 7.03 (d, J = 15.5 Hz, 1H), 6.94 (d, J = 11.3 Hz, 1H), 6.91 (d, J = 8.8 Hz, 2H), 3.84 (s, 3H). |
| | | | (2E,4E)-1-(1H-indol-3- | |
| | | | yl)-5-(4-methoxyphenyl) | |
| | | | penta-2,4-dien-1-one | |
| 12 | thiophene | F | 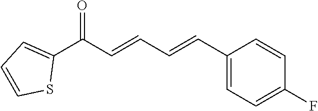 [SMILES: O=C(/C=C/C=C/c1ccc(F)cc1)c1cccs1] | δ 8.83 (s, 2H), 8.42-8.36 (m, 2H), 7.87 (d, J = 3.0 Hz, 2H), 7.45-7.39 (m, 2H), 7.30 (d, J = 1.4 Hz, 1H), 7.29 (s, 1H), 7.28 (d, J = 1.5 Hz, 1H). |
| | | | (2E,4E)-5-(4- | |
| | | | fluorophenyl)-1- | |
| | | | (thiophen-2-yl)penta-2,4- | |
| | | | dien-1-one | |
| 13 | thiophene | Br | 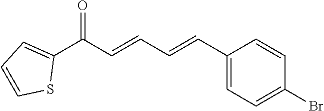 [SMILES: O=C(/C=C/C=C/c1ccc(Br)cc1)c1cccs1] | δ 7.79 (d, J = 3.8 Hz, 1H), 7.67 (d, J = 3.9 Hz, 1H), 7.64-7.57 (m, 1H), 7.50 (d, J = 8.5 Hz, 2H), 7.36 (d, J = 8.5 Hz, 2H), 7.19-7.15 (m, 1H), 7.01 (d, J = 14.7 Hz, 2H), 6.97 (d, J = 6.3 Hz, 1H). |
| | | | (2E,4E)-5-(4- | |
| | | | bromophenyl)-1- | |
| | | | (thiophen-2-yl)penta-2,4- | |
| | | | dien-1-one | |
| 14 | thiophene | OCH₃ | 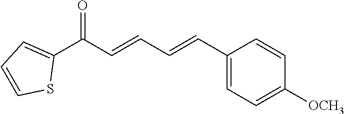 [SMILES: COc1ccc(/C=C/C=C/C(=O)c2cccs2)cc1] | δ 7.78 (d, J = 3.8 Hz, 1H), 7.68-7.58 (m, 2H), 7.45 (d, J = 8.7 Hz, 2H), 7.19-7.13 (m, 1H), 7.02-6.84 (m, 5H), 3.84 (s, 3H). |
| | | | (2E,4E)-5-(4- | |
| | | | methoxyphenyl)-1- | |
| | | | (thiophen-3-yl)penta-2,4- | |
| | | | dien-1-one | |
| 15 | 5-bromo- thiophene | H | 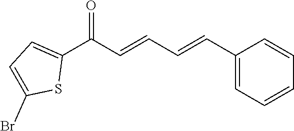 [SMILES: O=C(/C=C/C=C/c1ccccc1)c1ccc(Br)s1] | δ 7.62 (dd, J = 14.8, 10.1 Hz, 1H), 7.51 (dd, J = 9.1, 5.6 Hz, 3H), 7.41-7.32 (m, 3H), 7.13 (d, J = 4.0 Hz, 1H), 7.03 (dd, J = 18.8, 12.8 Hz, 2H), 6.89 (d, J = 14.8 Hz, 1H). |
| | | | (2E,4E)-1-(5- | |
| | | | bromothiophen-2-yl)-5- | |
| | | | pentylpenta-2,4-dien-1- | |
| | | | one | |
| 16 | 5-bromo- thiophene | F | 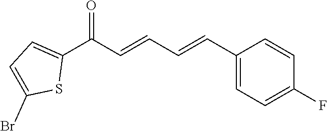 [SMILES: O=C(/C=C/C=C/c1ccc(F)cc1)c1ccc(Br)s1] | δ 7.60 (dd, J = 14.8, 10.8 Hz, 1H), 7.51 (d, J = 4.0 Hz, 1H), 7.48 (dd, J = 8.7, 5.4 Hz, 2H), 7.13 (d, J = 4.0 Hz, 1H), 7.07 (t, J = 8.6 Hz, 2H), 6.99 (d, J = 15.5 Hz, 1H), 6.90 (dd, J = 22.1, 12.8 Hz, 2H). |
| | | | (2E,4E)-1-(5- | |
| | | | bromothiophen-2-yl)-5- | |
| | | | (4-fluorophenyl)penta- | |
| | | | 2,4-dien-1-one | |
| 17 | 5-bromo- thiophene | Br | 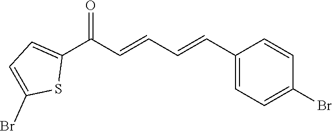 [SMILES: O=C(/C=C/C=C/c1ccc(Br)cc1)c1ccc(Br)s1] | δ 7.62-7.55 (m, 1H), 7.51 (d, J = 12.3 Hz, 3H), 7.36 (d, J = 8.5 Hz, 2H), 7.13 (d, J = 4.0 Hz, 1H), 6.98-6.95 (m, 2H), 6.89 (d, J = 14.8 Hz, 1H). |
| | | | (2E,4E)-5-(4- | |
| | | | bromophenyl)-1-(5- | |
| | | | bromothiophen-2- | |
| | | | yl)penta-2,4-dien-1-one | |
| 18 | 5-bromo- thiophene | OCH₃ | 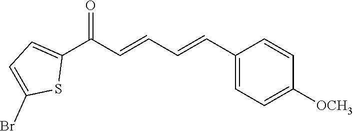 [SMILES: COc1ccc(/C=C/C=C/C(=O)c2ccc(Br)s2)cc1] | δ 7.62 (dd, J = 14.4, 11.1 Hz, 1H), 7.51 (d, J = 4.0 Hz, 1H), 7.44 (dd, J = 12.4, 6.4 Hz, 2H), 7.11 (dd, J = 12.5, 4.0 Hz, 1H), 7.00 (d, J = 15.5 Hz, 1H), 6.92-6.89 (m, 2H), 6.84 (d, J = 14.7 Hz, 2H), 3.84 (s, 3H). |
| | | | (2E,4E)-1-(5- | |
| | | | bromothiophen-2-yl)-5- | |
| | | | (4-methoxyphenyl)penta-2,4-dien-1-one | |

### Biological Assays

### Enzyme Assays and Inhibition Studies:

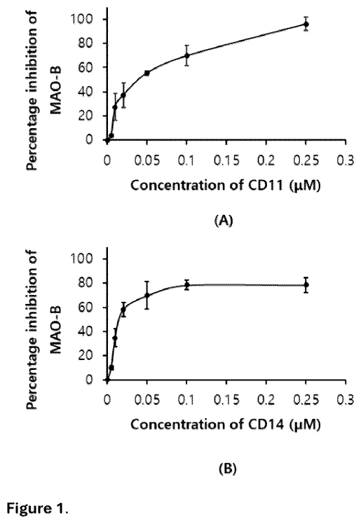

The inhibitory activities of MAO-A and MAO-B were assessed using kynuramine (0.06 mM) and benzylamine (0.3 mM) as substrates, respectively, following a standardized protocol^{[1]}. Absorbance readings were taken using a previously described method^{[2]}. The selectivity index (SI) of MAO-B was determined by the formula: (IC₅₀ of MAO-A)/(IC₅₀ of MAO-B). Test compounds were compared against standard compounds toloxatone and clorgyline for MAO-A inhibition and safinamide and pargyline for MAO-B inhibition. IC₅₀ values of lead compounds were measured at six different concentrations, while other effective compounds were assessed at three concentrations. Results were reported as mean±standard deviation (SD) of triplicate and duplicate experiments as provided in below Table 3. The percentage of inhibition is provided in FIG. 1.

TABLE 3
represents the IC₅₀ values of the compounds
of the present disclosure with MAO-A/B ^{a}.

| | Residual activity | | | | |
|---|---|---|---|---|---|
| Compounds | at 10 μM (%) | | IC₅₀ (μM) | | |
| No. (CD) | MAO-A | MAO-B | MAO-A | MAO-B | SI^{b} |
| 1 | 31.99 ± | 11.59 ± | 3.45 ± | 2.43 ± | 1.43 |
| | 1.88 | 1.31 | 0.07 | 0.70 | |
| 2 | 12.40 ± | −26.88 ± | 3.23 ± | 0.101 ± | 31.98 |
| | 1.62 | 0.40 | 0.24 | 0.02 | |
| 3 | 17.44 ± | 1.22 ± | 3.15 ± | 0.91 ± | 3.46 |
| | 0.711 | 0.22 | 0.10 | 0.19 | |
| 4 | 69.9 ± | −19.88 ± | 18.84 ± | 0.307 ± | 61.37 |
| | 18.04 | 0.45 | 0.70 | 0.05 | |
| 5 | 74.74 ± | 31.28 ± | >40 | 6.01 ± | >6.6 |
| | 12.4 | 5.81 | | 0.64 | |
| 6 | 86.79 ± | 27.17 ± | >40 | 3.46 ± | >11.56 |
| | 5.2 | 4.35 | | 0.31 | |
| 7 | 96.11 ± | 32.38 ± | >40 | 2.99 ± | >13.38 |
| | 2.77 | 2.28 | | 0.48 | |
| 8 | 86.92 ± | −21.5 ± | >40 | 0.230 ± | >173.91 |
| | 0.55 | 2.17 | | 0.01 | |
| 9 | 77.71 ± | 27.46 ± | >40 | 5.67 ± | >7.05 |
| | 2.29 | 0.76 | | 0.28 | |
| 10 | 50.66 ± | 74.69 ± | 12.73 ± | 17.76 ± | 0.72 |
| | 1.97 | 13.1 | 0.34 | 1.10 | |
| 11 | 43.57 ± | −17.61 ± | 6.58 ± | 0.063 ± | 104.44 |
| | 1.6 | 2.28 | 0.69 | 0.001 | |
| 12 | 52.27 ± | 47.59 ± | 12.98 ± | 11.51 ± | 1.13 |
| | 1.31 | 7.47 | 2.0 | 0.38 | |
| 13 | 68.77 ± | −5.27 ± | >40 | 0.136 ± | >294.12 |
| | 2.02 | 0.40 | | 0.008 | |
| 14 | 51.81 ± | −5.2 ± | 13.56 ± | 0.036 ± | 376.66 |
| | 2.92 | 1.7 | 0.65 | 0.008 | |
| 15 | 65.60 ± | −16.45 ± | 22.22 ± | 0.095 ± | 224.44 |
| | 3.87 | 0.29 | 3.92 | 0.006 | |
| 16 | 71.3 ± | −27.33 ± | 24.58 ± | 0.099 ± | 248.28 |
| | 1.33 | 2.0 | 0.95 | 0.02 | |
| 17 | 67.07 ± | −11.91 ± | 25.63 ± | 0.40 ± | 258.38 |
| | 6.73 | 0.33 | 11.03 | 0.07 | |
| 18 | 59.64 ± | −9.3 ± | 19.82 ± | 0.76 ± | 26.07 |
| | 8.73 | 2.55 | 5.08 | 0.02 | |
| Safinamide | — | — | — | 0.021 ± | — |
| | | | | 0.002 | |
| Pargyline | — | — | — | 0.137 ± | — |
| | | | | 0.01 | |
| Toloxatone | — | — | 1.77 ± | — | — |
| | | | 0.22 | | |
| Clorgyline | — | — | 0.0077 ± | — | — |
| | | | 0.0006 | | |

^{a} Results were expressed as mean ± standard deviation (SD) of duplicate or triplicate experiments;
^{b}SI values were expressed for MAO-B, compared with MAO-A, based on their IC₅₀ values.

### Enzyme and Inhibition Kinetics:

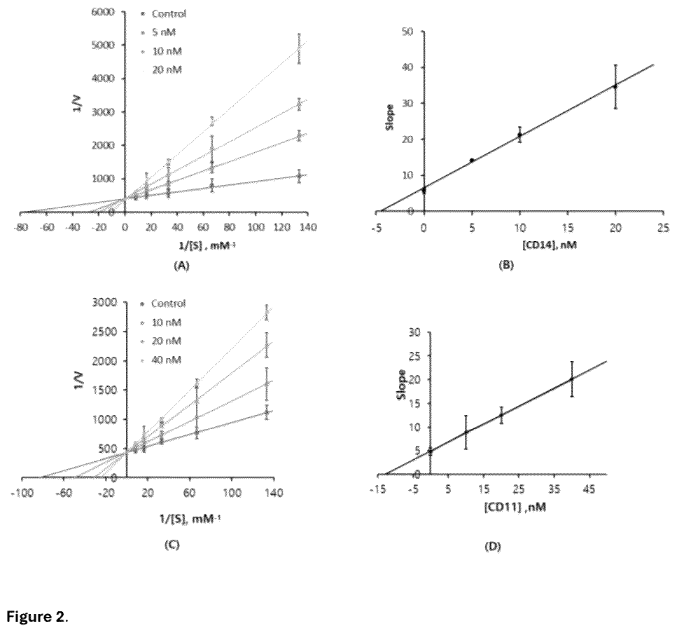

The enzyme kinetics of the two potent compounds 11 and 14 were investigated at five different concentrations of benzylamine (ranging from 0.0375 to 0.60 μM) for MAO-B^{[1], [3]}, and at three different concentrations of compound 11 (10, 20, and 40 nM) and compound 14 (5, 10, and 20 nM). Enzyme inhibition patterns and Ki values were determined by comparing the Lineweaver-Burk plots and their respective secondary plots^{[2]}. The relevant results are provided in FIG. 2.

### Reversibility Studies

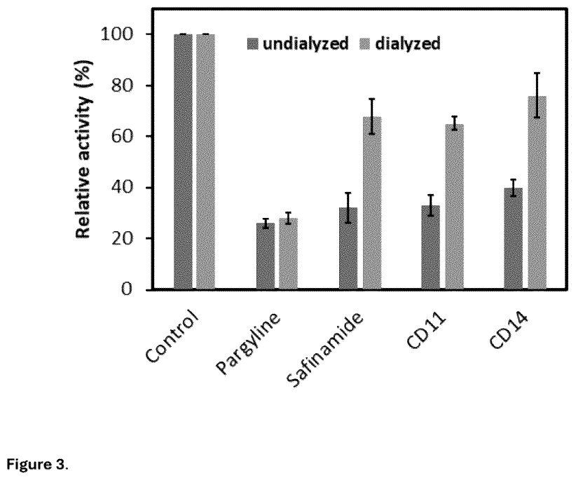

The reversibility of the compounds was assessed following a standardized protocol^{[1], [3]}. Residual activities of undialyzed (A_{U}) and dialyzed (A_{D}) were analyzed at a concentration approximately 2.0 times the IC₅₀ values after 30 minutes of preincubation. It is demonstrated in FIG. 3. The recovery of enzyme activities was compared with those of the standard compound's safinamide and pargyline (representing reversible and irreversible inhibitors, respectively) of MAO-B. Reversibility patterns were determined by comparison with control samples^{[1][3]}. These results are provided in the above Table 3. It illustrates that all eighteen compounds exhibited inhibition of Monoamine oxidase B.

Among the analyzed compounds, Compounds 11 and 14 revealed the significant inhibitory potential towards MAO-B with IC₅₀ values of 0.063±0.001 μM and 0.036±0.008 μM, respectively. Therefore, these compounds are more effective as MAO-B inhibitors compared to the reference drug pargyline (IC₅₀=0.137±0.01 μM). Whereas compounds 1, 2, and 3 demonstrated comparable inhibitory activities towards MAO-A with IC₅₀ values of 3.45±0.07, 3.23±0.24, and 3.15±0.10 μM, respectively. These results showed that analyzed compounds were highly selective towards MAO-B.

Further, kinetics analysis and reversibility test demonstrated that both compound 11 and compound 14 were competitive reversible MAO-B with Kᵢ values of 12.67±3.85 nM and 4.5±0.62 nM, respectively. Additionally, these Compounds 11 and 14 also had high selectivity index (SI) values for MAO-B (104.44 and 376.66) respectively. These results indicate that Compounds 11 and 14 are potent selective MAO-B inhibitors.

### Advantages

The present disclosure pertains to a class of compounds aiding as potent inhibitors of Monoamine oxidase B (MAO-B). These compounds are characterized by their notably high selectivity index (SI) values for MAO-B and their competitive, reversible inhibition of the enzyme. Such attributes take the compounds as promising candidates for therapeutic intervention in neurological and neurodegenerative disorders, thereby emphasising their potential significance in pharmaceutical research and drug development.

The foregoing description of the various embodiments is provided to enable any person skilled in art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein, and instead the claims should be accorded the widest scope consistent with the principles and novel features disclosed herein.

### REFERENCES

1. G. S. Jeong, M. G Kang, J. Y. Lee, S. R. Lee, D. Park, M. Cho, H. Kim. Inhibition of butyrylcholinesterase and human monoamine oxidase-B by the coumarin glycerol and liquiritigenin isolated from Glycyrrhiza uralensis. Molecules 25 (17), 2020, 3896.
2. N. A. Rehuman, J. M. OH, L. R. Nath, A. Khames, M. A. Abdelgawad, N. Ambacorta, O. Nicolotti, R. K. Jat, H. Kim, B. Mathew. Halogenated coumarin-chalcones as multifunctional monoamine oxidase-B and butyrylcholinesterase inhibitors, ACS omega 6 (42) 2021, 28182-28193.
3. H. W. Lee, H. W. Ryu, M. G. Kang, D. Park, S. R. Oh, H. Kim. Potent selective monoamine oxidase B inhibition by maackiain, a pterocarpan from the roots of Sophora flavescens, Bioorganic & Medicinal Chemistry Letters, 26 (19) 2016, 4714-4719.

What is claimed is:

1. A compound of formula (I): 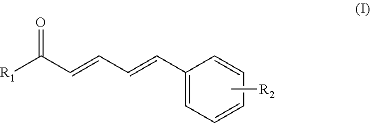
or a pharmaceutically acceptable salt thereof;
wherein
R₁ is selected from a group comprising C₆-C₁₄ aryl, 5-14 membered heterocyclyl, 5-14 membered heteroaryl, wherein aryl, heterocyclyl, and heteroaryl, are optionally substituted with a group selected from hydrogen, halogen, C₁-C₆ alkyl, C₁-C₆ alkoxy, and —NO₂; and
R₂ is selected from a group comprising hydrogen, halogen, C₁-C₆ alkyl, C₁-C₆ alkoxy, and —NO₂.

2. The compound as claimed in claim 1, wherein the R₁ is thiophene, indole, benzofuran, or benzodioxol.

3. The compound as claimed in claim 1, wherein the R₁ is substituted with halogen.

4. The compound as claimed in claim 1, wherein the halogen is selected from fluorine, chlorine, bromine, and iodine.

5. The compound as claimed in claim 1, wherein the alkyl is selected from a group comprising methyl, ethyl, propyl, butyl, pentyl, and hexyl.

6. The compound as claimed in claim 1, wherein the alkoxy is selected from a group comprising methoxy, ethoxy, propoxy, and butoxy.

7. The compound as claimed in claim 1, wherein the R₂ is hydrogen.

8. The compound as claimed in claim 1, wherein the R₂ is halogen.

9. The compound as claimed in claim 1, wherein the R₂ is alkoxy.

10. The compound as claimed in claim 1, wherein the R₂ is —NO₂.

11. The compound as claimed in claim 1, wherein the compound has the following Formula (Ia): 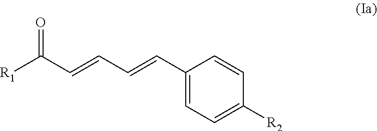
or a pharmaceutically acceptable salt thereof.

12. The compound as claimed in claim 1, wherein the compound is selected from the following:
(2E,4E)-1-(benzo[d][1,3]dioxol-5-yl)-5-(4-nitrophenyl) penta-2,4-dien-1-one (compound 1);
(2E,4E)-1-(benzo[d][1,3]dioxol-5-yl)-5-(4-bromophenyl) penta-2,4-dien-1-one (compound 2);
(2E,4E)-1-(benzo[d][1,3]dioxol-5-yl)-5-(4-methoxyphenyl) penta-2,4-dien 1-one (compound 3);
(2E,4E)-1-(benzofuran-2-yl)-5-phenylpenta-2,4-dien-1-one (compound 4);
(2E,4E)-1-(benzofuran-2-yl)-5-(4-nitrophenyl) penta-2,4-dien-1-one (compound 5);
(2E,4E)-1-(benzofuran-2-yl)-5-(4-bromophenyl) penta-2,4-dien-1-one (compound 6);
(2E,4E)-1-(benzofuran-2-yl)-5-(4-methoxyphenyl) penta-2,4-dien-1-one (compound 7);
(2E,4E)-1-(benzofuran-2-yl)-5-(4-fluorophenyl) penta-2,4-dien-1-one (compound 8);
(2E,4E)-5-(4-fluorophenyl)-1-(1H-indol-3-yl) penta-2,4-dien-1-one (compound 9);
(2E,4E)-5-(4-bromophenyl)-1-(1H-indol-3-yl) penta-2,4-dien-1-one (compound 10);
(2E,4E)-1-(1H-indol-3-yl)-5-(4-methoxyphenyl) penta-2,4-dien-1-one (compound 11);
(2E,4E)-5-(4-fluorophenyl)-1-(thiophen-2-yl) penta-2,4-dien-1-one (compound 12);
(2E,4E)-5-(4-bromophenyl)-1-(thiophen-2-yl) penta-2,4-dien-1-one (compound 13);
(2E,4E)-5-(4-methoxyphenyl)-1-(thiophen-3-yl) penta-2,4-dien-1-one (compound 14);
(2E,4E)-1-(5-bromothiophen-2-yl)-5-phenylpenta-2,4-dien-1-one (compound 15);
(2E,4E)-1-(5-bromothiophen-2-yl)-5-(4-fluorophenyl) penta-2,4-dien-1-one (compound 16);
(2E,4E)-5-(4-bromophenyl)-1-(5-bromothiophen-2-yl) penta-2,4-dien-1-one (compound 17); and
(2E,4E)-1-(5-bromothiophen-2-yl)-5-(4-methoxyphenyl) penta-2,4-dien-1-one (compound 18).

13. A pharmaceutical composition comprising the compounds as claimed in claim 1 and a pharmaceutically acceptable carrier.

14. A pharmaceutical composition comprising the compounds as claimed in claim 12 and a pharmaceutically acceptable carrier.

15. A process of preparing compounds as claimed in claim 1, wherein the process comprises a step of reacting a substituted cinnamaldehyde of formula (A) with a heterocyclic acetophenone of formula B in a solvent, in the presence of a catalyst, at room temperature:

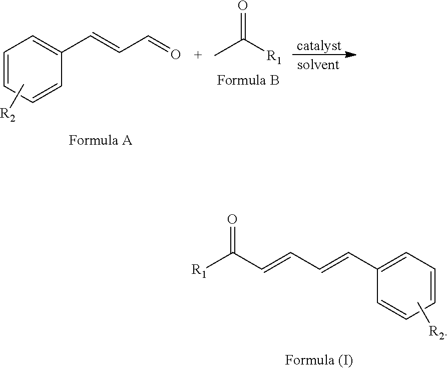

16. The process as claimed in claim 15, wherein the catalyst is pyrrolidine, and the solvent is selected from ethanol and methanol.

17. A method of treating or preventing a disease, disorder or condition in a subject comprising administering to the subject a therapeutically effective amount of a compound of formula (I) as claimed in claim 1.

18. The method as claimed in claim 17, wherein the disease, disorder or condition is a neurological disorder or a neurodegenerative disorder.

19. The method as claimed in claim 18, wherein the neurological disorder or the neurodegenerative disorder is selected from the group comprising amyotrophic lateral sclerosis, multiple sclerosis, Parkinson's disease, Alzheimer's disease, Huntington's disease, multiple system atrophy, tauopathies, and prion diseases.

20. The method as claimed in claim 19, wherein the neurological disorder or the neurodegenerative disorder is Parkinson's disease (PD).
